SEQUENCE LISTING (7 sequences)

SEQ ID NO: 1 - DNA, 18 bp, Corynebacterium glutamicum
gctccagccacccaaaac

SEQ ID NO: 2 - DNA, 47 bp, Corynebacterium glutamicum
ggcttcatcgacagtctgacgaccgactcaaccactaatgcgtcgtc

SEQ ID NO: 3 - DNA, 20 bp, Artificial
  DNA oligonucleotide with 6-FAM as fluorescence dye at its 5′ end and BHQ1 as quencher at its 3′ end
ggcttcatcgacagtctgac

SEQ ID NO: 4 - DNA, 20 bp, Corynebacterium glutamicum
gtcagactgtcgatgaagcc

SEQ ID NO: 5 - DNA, 20 bp, Escherichia coli
atgctacccctgaaaaactc

SEQ ID NO: 6 - DNA, 47 bp, Escherichia coli
tttacttctttgcgttatgtctctgactcgcttgaactgatttcctc

SEQ ID NO: 7 - DNA, 18 bp, Artificial
  DNA oligonucleotide with 6-FAM as fluorescence dye at its 5′ end and BHQ1 as quencher at its 3′ end
tttacttctttgcgttat

(19) United States
(12) Patent Application Publication
(10) Pub. No.: US 2012/0149011 A1
(43) Pub. Date: Jun. 14, 2012

(54) METHOD FOR DETECTING TARGET NUCLEIC ACIDS

(75) Inventors: Ralf Himmelreich, Langenfeld (DE); Gerd Grosshauser, Pulheim (DE); Thomas Rothmann, Langenfeld (DE)

(73) Assignee: QIAGEN GMBH, Hilden (DE)

(21) Appl. No.: 13/390,887

(22) PCT Filed: Aug. 16, 2010
(86) PCT No.: PCT/EP2010/005019

§ 371 Date: Feb. 16, 2012

(30) Foreign Application Priority Data
Aug. 21, 2009 (EP) 09010757.4

(51) Int. Cl. C12Q 1/68; G01N 25/04; C12Q 1/70; G01N 21/64

(52) U.S. Cl. 435/5; 435/6.12; 435/6.11

(57) ABSTRACT

The invention relates to a method for detecting target nucleic acids which are detected by means of a specific sequence tag which is not part of the target nucleic acid.

The present invention relates to a method for detecting target nucleic acids, wherein the target nucleic acids are detected by means of a specific sequence tag which is not part of the target nucleic acid.

The detection of target nucleic acids by primer-mediated amplification has been widespread for many years in molecular biology. These technologies include the polymerase chain reaction (PCR), rolling circle amplification (RCA), strand displacement amplification (SDA), multiple displacement amplification (MDA), strand displacement cascade amplification (SDCA), self-sustained sequence replication (3SR), nucleic acid based amplification (NASBA), amplification by means of Qβ replicase and other linear amplification techniques such as for example cycle sequencing.

By means of the PCR and modifications thereof, defined DNA sequences, even from a mixture of very different sequences, can be greatly multiplied by use of sequence-specific oligonucleotides, so-called primers. This multiplication, also described as amplification, takes place by means of a DNA polymerase. DNA polymerases form double-stranded DNA from single-strand DNA by attaching to the free 3′-OH ends of a DNA fragment already attached to the single strand the respective bases complementary to the remaining single strand. Since DNA polymerases are not sequence-specific, filling in of a single-stranded region on a DNA sequence always takes place when partial double strand formation, e.g. through attachment of a DNA fragment to the single strand, is present. In the PCR, sequence-specific primers which are complementary to the sequence which is to be multiplied are added to the DNA from which a defined sequence is to be amplified. By means of suitable primers, it is also possible to incorporate nucleotide sequences which were not present in the original nucleotide sequence of the target nucleic acid into an amplification product. For this purpose, at the 5′ end of the primer, nucleotide sequences are selected which are not complementary with the target nucleic acid. Thereby it is for example possible to incorporate cleavage sites for restriction endonucleases or recognition sequences for other nucleic acid-binding proteins which had not been present in the original target nucleic acid into the amplification products of a target nucleic acid.

The PCR is a simple and flexible method for the reproduction and limited modification of the nucleotide sequence of a target nucleic acid, which is by far the most commonly used for amplification in molecular biology. However, in order to achieve the amplification, a protocol which passes through at least two, but very often three, different temperature steps in 20 to 40 cycles must be executed. This necessitates specialized instrumentation and a relatively time-consuming process, since only a doubling of the target nucleic acid is possible in each cycle.

Rolling circle amplification (RCA), mediated by a DNA polymerase with strand displacement activity and a lack of 5′-3′ exonuclease activity, can replicate circular oligonucleotides under isothermal conditions. When a single primer is used, within a few minutes the RCA forms a single-strand linear chain of hundreds or thousands of tandem DNA copies of a target nucleic acid which are covalently bound to this target nucleic acid. The formation of a linear amplification product allows both the spatial resolution and also the precise quantification of a target nucleic acid. The DNA which is formed through the RCA can be labeled by fluorescent oligonucleotide tags which can hybridize at many sites in the tandem DNA sequences, since the sequence of the target nucleic acid constantly repeats. The RCA can be performed in solution, in situ and in microarrays. In solid phase formats, the RCA is sufficiently sensitive to be able to detect a single molecule of a target nucleic acid.

A more complex modification of RCA is RCA with a pair of different primers. This is described as hyperbranched, ramification or cascade RCA. As in linear RCA, one primer is complementary to the target nucleic acid, whereas the second primer can bind to the single-strand DNA regions which have arisen through the primary RCA product. Subsequently in this case the RCA proceeds as a chain reaction with a cascade of a large number of hybridizations, primer elongations and strand displacements, in which both primers are involved. The result of this reaction are concatemeric double-stranded DNA fragments. Through this type of RCA up to about 10⁹ copies of the target nucleic acid can be produced within one hour.

Both in the PCR with its modifications and also in the RCA with its modifications, the detection of the target nucleic acid takes place via the amplification of a part of the target nucleic acid.

A further method for detecting target nucleic acids is nicking endonuclease signal amplification (NESA), in which the signal amplification is effected via short oligonucleotides. NESA is a sensitive method for the detection of specific DNA, in which the signal is amplified by means of a nicking endonuclease. Double-stranded DNA which possesses a recognition site for a nicking endonuclease is denatured. Next, a fluorescence-labeled complementary oligonucleotide present in excess is hybridized onto this target nucleic acid, so that the labeled oligonucleotide together with one strand of the target nucleic acid contains a recognition site for a nicking endonuclease. This nicking endonuclease cleaves the labeled oligonucleotide, but leaves the target nucleic acid intact. Since due to the cleavage by the endonuclease both oligonucleotides are now shorter than the original uncleaved labeled oligonucleotide, their affinity to the target nucleic acid at the set experiment temperature is no longer sufficient, so that both of the short oligonucleotides, one of which still possesses the fluorescence labeling, dissociate from the target nucleic acid. After the dissociation of both oligonucleotides from the target nucleic acid, the target nucleic acid can hybridize with a further still uncleaved fluorescence-labeled oligonucleotide, and a new cycle is initiated. The detection of the target nucleic acid is effected via the short fluorescence-labeled oligonucleotides obtained in this process. For this purpose, after completion of the reaction, the reaction mixture is subjected to a capillary electrophoresis, in which the shorter fluorescence-labeled oligonucleotides can be distinguished from the uncleaved longer ones. Compared to the PCR, this method for detecting target nucleic acids is considerably faster; the reaction time is between 10 and 30 mins depending on the concentration of the target nucleic acid. In addition in each case a further ca. 10 mins must be reckoned for the denaturation of the double-strand nucleic acid and ca. 10 mins for the capillary electrophoresis.

A further method for detecting target nucleic acids by means of a cleavage site for an endonuclease is the exponential amplification reaction (EXPAR) and the linear amplification modification thereof. In the linear modification, a complementary oligonucleotide with a recognition sequence for a nicking endonuclease hybridizes onto a single-strand target nucleic acid. After the nicking, the oligonucleotide now consists of two shorter oligonucleotides which are bound onto the target nucleic acid. The experimental conditions, the length of the two cleaved oligonucleotides and the reaction temperature, are selected such that the shorter oligonucleotide, but not the longer one, dissociates from the target nucleic acid. The longer oligonucleotide, which has remained on the target nucleic acid, now serves as a primer, so that the single-strand region of the target nucleic acid is again filled in. In the next cycle, the nicking endonuclease again cleaves the single strand, and a further short oligonucleotide dissociates from the target nucleic acid. The detection of the target nucleic acid is effected by mass spectrometry, via the short oligonucleotides formed in this reaction.

In the exponential modification, the dissociating oligonucleotide of the linear amplification is used in order to form a new primer which can bind to a so-called amplification template. This amplification template is added to the reaction mixture in addition to the target nucleic acid. The amplification template possesses a recognition site for the nicking endonuclease. In addition, the dissociating oligonucleotide can bind both at the 5′ and also at the 3′ end. In the first step, the oligonucleotide dissociated in the previous cycle binds to the complementary sequence which lies 5′ from the recognition site for the nicking endonuclease. Admittedly this binding event is relatively rare, since the reaction conditions are selected such that the oligonucleotide dissociates from its complementary sequence rather than binds to it, however this weak binding affinity nonetheless suffices to form a transient complex between the dissociated oligonucleotide and the amplification template. In the second step, the oligonucleotide, which now serves as a primer, is elongated at its 3′ end over the whole amplification template. A double-strand amplification template is thus formed which possesses a recognition site for the nicking endonuclease and after cleavage by the nicking endonuclease once again releases a short dissociating oligonucleotide which once again can bind to a further amplification template. In this method also, the detection of the dissociated short oligonucleotides is effected by means of mass spectrometry.

Both NESA and also EXPAR utilize the properties of a nicking endonuclease for the generation of oligonucleotides which can then be specifically detected. Both reactions have the advantage compared to a PCR that they can proceed under isothermal conditions and that the reaction takes place very rapidly.

However, both methods have considerable disadvantages which markedly limit their universal usability. Thus both NESA and also EXPAR are limited in the choice of possible sequences for the target nucleic acids to be detected. For both methods, it is absolutely necessary that a recognition site for a nicking endonuclease is already present on the target nucleic acid. Since however there are only very few different nicking endonucleases which recognize different nucleotide sequences, there are only very few naturally occurring nucleic acids which contain a recognition site for a nicking endonuclease in their sequence. Thus it is not possible to detect the majority of the naturally occurring nucleic acids directly by means of NESA or EXPAR.

Furthermore, the choice of experimental conditions and the choice of the oligonucleotides which can be used for the specific detection are very severely limited. Firstly, the two halves into which the oligonucleotide is cleaved by the nicking endonuclease must differ markedly in their affinity for the target nucleic acid, so that it is ensured that only the shorter of the two oligonucleotide halves, but not the longer, dissociates from the target nucleic acid. This necessitates a defined narrow temperature range for conducting the experiment, which is however not inevitably compatible with the temperature optima of the enzymes involved in the reaction. The consequence of this is that from these viewpoints also both NESA and also EXPAR are not universally usable.

Secondly, a multiplex approach is also only possible to a very limited extent owing to the fact that these oligonucleotides must display complete sequence complementarity to the target nucleic acid. Admittedly in the case of NESA the oligonucleotides can be labeled with fluorescent dyes in various ways, however there are only a limited number of dyes which can be detected at different wavelengths, so that the multiplex properties are severely limited. In the case of EXPAR, a multiplexing is only possible via the choice of oligonucleotides of different length which dissociate from the target nucleic acid, as a result of which the number of analyses of target nucleic acids is very severely limited, especially since in addition a temperature range must also still be found in which all shorter oligonucleotide halves obtained from the oligonucleotides cleaved by the nicking endonuclease, which must all have a different length, dissociate from the target nucleic acid, but the longer ones must remain on this. If an exponential detection by EXPAR is to be possible, in addition all these short oligonucleotide halves must also be able to form a transient complex with an amplification template with equal efficiency, in order to be able to exclude a falsification of the quantification, which is experimentally only possible with great difficulty. Hence both EXPAR and also NESA can in practice scarcely be used for a multiplexing approach in which several target nucleic acids can be detected simultaneously in parallel.

The purpose of the present invention is to overcome the disadvantages known from the state of the art and to provide a method for detecting target nucleic acids which makes it possible also to detect more than one target nucleic acid in the parallel approach by multiplexing and in which the nucleotide sequence of the target nucleic acid does not have to have a recognition sequence for an endonuclease.

This problem is solved by a method for detecting target nucleic acids comprising the following process steps:

a) primer-mediated amplification of at least one target nucleic acid, wherein at least one of the primers used for the amplification is a sequence tag primer which has a non-hybridizing part at its 5′ end, wherein this non-hybridizing part has a first sequence which during the amplification creates a cleavage site for a nicking endonuclease on the newly synthesized strand complementary to it, and furthermore 5′ from this first sequence has a second sequence which during the amplification creates a sequence tag on the newly synthesized strand complementary to it;

b) contacting of the at least partly double-stranded amplification product from step a) with nucleotides, a nicking endonuclease and a polymerase, wherein the polymerase has a strand displacement activity and no 5′→3′ exonuclease activity;

c) isothermal amplification of the sequence tag created in step a) by single or multiple repetition of a cycle having the following steps:
i) insertion of a nick at the cleavage site inserted in step a) by means of the endonuclease from step b);
ii) filling of the nick beginning at the free 3′ end created in step i) with complementary nucleotides by means of the polymerase from step b) with simultaneous displacement of the sequence tag from the double strand.

d) specific detection of the sequence tag amplified in step c).

With the method according to the invention one or more than one target nucleic acid can be detected. The target nucleic acid or acids which can be detected by means of the method according to the invention can be DNA, RNA or a mixture thereof. In a preferred embodiment, DNA is detected. The DNA can in each case be one or more cDNAs, genomic DNAs or a fragment thereof, plasmid DNAs or a fragment thereof, viral DNAs or a fragment thereof, mitochondrial DNAs or a fragment thereof, plastid DNAs or a fragment thereof, or the combination of two or more of these DNAs.

The target nucleic acids which are detected by the method according to the invention do not have to have any specific nucleotide sequences which serve as a recognition and/or binding site for nicking endonucleases, and moreover also need no specific sequence or structural motifs. Target nucleic acids which differ only in one nucleotide in their sequence can be specifically detected by the method according to the invention. Advantageously therefore, any specific nucleic acid can be detected irrespective of its nucleotide sequence.

In a first reaction step a), the target nucleic acid is amplified with primer mediation. During this, at least one of the primers used is a sequence tag primer. A sequence tag primer is characterized in that at its 5′ end it has a part which does not hybridize to the target nucleic acid. This non-hybridizing part has a first sequence which during the amplification creates a cleavage site for a nicking endonuclease on the newly synthesized strand complementary to it. Furthermore, this non-hybridizing part 5′ from this first sequence has a second sequence which during the amplification creates a sequence tag on the newly synthesized strand complementary to it. Consequently, after the amplification, the amplified target nucleic acid has an at least partly double-stranded region which as well as the sequence of the target nucleic acid has on the reverse strand a first sequence which serves as a cleavage site for a nicking endonuclease and also a second sequence which has a sequence independent of the target nucleic acid and is subsequently used as a sequence tag. The sequence tag is thus a sequence which is complementary to the 5′ end of the sequence tag primer—namely 5′ to the endonuclease cleavage site. Through the amplification of the target nucleic acid in step a) with the primers according to the invention, at least one of which is a sequence tag primer, the amplification products now have in their nucleotide sequence additionally to the nucleotide sequence of a part region or the whole sequence of the target nucleic acid at the 3′ end of the reverse strand, firstly a cleavage site for a nicking endonuclease and furthermore 3′ therefrom a sequence tag which is subsequently used as a recognition sequence characterizing the target nucleic acid. The sequence tag is specifically detected in step d) of the method according to the invention. The specific detection of the sequence tag in step d) thus effects the specific detection of the target nucleic acid.

The nucleotide sequence of the sequence tag is freely selectable; it must only in the sense of the present invention be created such that it cannot hybridize to the target nucleic acid(s) or to another nucleic acid which is present in the reaction mixture during the amplification.

In a preferred embodiment, the sequence tag has a length of at least 12 nucleotides, especially preferably of at least 15 nucleotides, and quite especially preferably the sequence tag has a length of 18 to 30 nucleotides.

In a further preferred embodiment, the sequence tag has a GC content of 40 to 60%, and has a melting temperature of 48 to 74° C.

In a further preferred embodiment, the sequence tag has no sequence motifs forming dimers or hairpins.

In a preferred embodiment, two or more target nucleic acids are amplified in step a) of the method according to the invention. This amplification can be performed in the same reaction vessel as a multiplex amplification, but it can also be performed in the parallel approach in spatially separate reaction vessels. For each target nucleic acid, in each case at least one specific sequence tag primer is preferably used for the amplification.

In an especially preferred embodiment, the cleavage site for a nicking endonuclease is identical for each of the sequence tag primers used, so that in the multiplexing approach only one species of nicking endonuclease is needed, which is capable of placing a nick exclusively on the reverse strand of all target nucleic acids amplified in step a).

On the other hand, in an especially preferred embodiment, the sequence of the sequence tag differs for each sequence tag primer, so that each target nucleic acid which is amplified in step a) in the multiplexing approach or in the parallel approach is characterized by a different sequence tag, which is used as the respective recognition sequence characterizing the target nucleic acid. Thus, according to the invention, for each target nucleic acid, a sequence tag primer which creates a specific sequence of the sequence tag is used for the amplification. The detection of the sequence tag here can take place in the parallel approach in more than one reaction vessel or advantageously in the multiplexing approach in one reaction vessel.

For the primer-mediated amplification of the target nucleic acid, isothermal amplification methods, which are known to those skilled in the art from the state of the art, are suitable. These include for example iCNA (Takara), tHDA (BioHelix) and RPA (TwistDx).

In a preferred embodiment, the primer-mediated amplification of the target nucleic acid is effected in step a) by polymerase chain reaction (PCR). Various embodiments of the PCR are known from the state of the art, and familiar to those skilled in the art.

In an especially preferred embodiment, the amplification of the target nucleic acid takes place by means of PCR with the aid of nested primers. In this method well-known from the state of the art, firstly a part region of the target nucleic acid is multiplied with an outer primer pair. This part region then serves as a template for a second amplification with a second primer pair, the primers whereof each bind 3′ from the respective primer of the first primer pair and thus further amplify an “inner region” of the template. Through this manner of amplification of the target nucleic acid in two steps, a higher specificity of multiplication is achieved. In the PCR with the aid of nested primers, at least one primer of the second inner primer pair is a sequence tag primer.

After the amplification, the primer-mediated amplification product can be present entirely as a double strand, but also be only partly double-stranded. In this case, as well as a double-stranded region, the amplification product has one or more other regions in which the amplification product is present as a single strand. On the other hand, in the method according to the invention it is essential that the region of the amplification product which is recognized and cleaved by the nicking endonuclease is present as a double strand.

In step b), a nicking endonuclease recognizes the recognition sequence on the double strand of the amplification product and depending on the nature of the nicking endonuclease cleaves the reverse strand to the sequence tag primer in or next to this recognition sequence, as a result of which a so-called nick is inserted into the double-stranded amplification product, in which the 5′-3′ phosphodiester bond between two nucleotides is hydrolytically cleaved. The nicking endonuclease thus acts as a phosphodiesterase, so that a single-strand break is inserted in the double strand and a free 3′-OH end is created, which serves as an attachment point for a polymerase. In the process according to the invention, only one strand of the double strand is always cleaved, namely the reverse strand, on which the recognition sequence for the nicking endonuclease is also situated, and the other strand remains intact. Endonucleases which cleave not only one strand of the double strand, but instead both, are not suitable for the method according to the invention.

Examples of suitable nicking endonucleases are Nt.BstNBI, Nt.BspQI, Nb.BbvCI, Nb.BsmI, Nb.BsrDI, Nb.BtsI, Nt.AlwI, Nt.BbvCI, Nt.CviPII, Nt.BsmAI, Nb.Bpu10I and Nt.Bpu10I. Further suitable nicking endonucleases are familiar to those skilled in the art.

The nick in the double-stranded amplification product is recognized by a polymerase which possesses no 5′→3′ exonuclase activity and also has a strand displacement activity. A polymerase in the sense of the present invention is an enzyme for nucleic acid replication and/or for nucleic acid repair. The polymerase fills the nick at the 3′-OH end beginning with nucleotides which are complementary to the template strand. For this a (desoxy)-ribonucleotide phosphate corresponding to the complementary base is successively attached each time and incorporated via a phosphodiester bond with elimination of pyrophosphates. The polymerization reaction always takes place in the 5′→3′ direction. During this process, the sequence tag is displaced from the amplified nucleic acid through the strand displacement activity of the polymerase and is present unhybridized as a single strand.

Suitable polymerases are all polymerases which possess a strand displacement activity and at the same time no 5′→3′ exonuclease activity.

The polymerase is preferably a DNA polymerase which fills the nick with desoxyribo-nucleotides. These for example include Vent exo⁻, Deep Vent exo⁻, Bst exo⁻, Klenow fragment of DNA polymerase I, Phi 29 DNA polymerase and 9° Nm DNA polymerase. Further suitable DNA polymerases are familiar to those skilled in the art.

In a preferred embodiment, the temperature optima for the enzymatic activity of the nicking endonuclease and the polymerase lie in a similar temperature range, and an example of such a combination is the combination of the nicking endonuclease Nt.BstNBI with the DNA polymerase Vent exo⁻. Further preferred combinations readily follow from the comparison of the temperature optima of a nicking endonuclease with a polymerase. These temperature optima are also familiar to those skilled in the art.

After the nick has been recognized by the polymerase in step c) of claim 1 and filled with the nucleotides complementary to the amplification product, whereby the sequence tag has been displaced from the amplification product, the amplification product is now again present in this region as an intact double strand and again possesses a cleavage site and a recognition sequence for a nicking endonuclease and also a sequence tag. The amplification product thus now again has the same nucleic acid sequence in this region as before the introduction of the nick by the nicking endonuclease, so that the procedure of insertion of a nick by the nicking endonuclease, filling of the nick by the polymerase and displacement of the sequence tag can be repeated any number of times, so that the sequence tag is isothermally amplified. Consequently, only the sequence which is complementary to the 5′ end of the primer is filled by the polymerase.

The quantity of the isothermally amplified and released sequence tags from step c) can be modulated by several factors. The reaction time during which the isothermal amplification of the sequence tag with the steps of claim 1) c) i) and ii) proceeds has a major influence on the quantity attained. The longer the reaction time, the more sequence tags can normally be formed and released.

A further important factor is the reaction temperature in step c). As in all enzymatic reactions, the more the reaction temperature coincides with the temperature optimum for the activity of the enzymes involved, the faster the conversion takes place. Consequently, the more similar the temperature optima of the enzymes involved, the nicking endonuclease and the polymerase are, and the more precisely the reaction temperature coincides with these temperature optima, the more efficient is the isothermal amplification of the sequence tag.

The choice of the reaction temperature also plays a significant part in the amplification of the sequence tag in another respect. If the reaction temperature lies below the melting temperature of the sequence tag onto the amplification product from step a), then after insertion of the nick the sequence tag can only be released by a polymerase with strand displacement activity. If the reaction temperature lies above the melting temperature of the sequence tag onto the amplification product, then this hybrid is so unstable after insertion of the nick that the sequence tag can be released even without a polymerase with strand displacement activity. In this case, the sequence tag is not actively displaced, but instead released passively.

In a preferred embodiment, the reaction temperature in step c) is selected such that the sequence tag is released through the strand displacement activity of a polymerase, in order to ensure that in parallel with the release of the sequence tag the nick has been filled again with complementary nucleotides.

In an especially preferred embodiment, the reaction temperature in step c) is selected such that the sequence tag is released through the strand displacement activity of a polymerase and that the reaction temperature coincides with the temperature optima of the nicking endonuclease and the polymerase.

Other factors which influence the quantity of the sequence tag include the quantity of amplified target nucleic acid, the quantity of nucleotides and the buffer conditions. Further factors are familiar to those skilled in the art.

In step d) of the method according to the invention, the sequence tags isothermally amplified in step c) are specifically detected by means of a probe which is targeted on the sequence tag.

Consequently, the probe must be complementary to the sequence tag at least in one part region and be able to hybridize with this.

The probe is preferably an oligonucleotide, and alternative solutions are familiar to those skilled in the art.

Here the probe can possess different modifications, such as for example fluorescent dyes, quantum dots or gold nanoparticles. Other possibilities for modification are familiar to those skilled in the art.

In a preferred embodiment, the probe has a length of at least 12 nucleotides, especially preferably of at least 15 nucleotides, and quite especially preferably the probe has a length of 18 nucleotides to 30 nucleotides.

In a further preferred embodiment, the probe has a GC content of 40 to 60%, and has a melting temperature of 48 to 74° C.

Possibilities for the detection of the hybrid of sequence tag and probe are familiar from the state of the art to those skilled in the art. In one embodiment, the probe is labeled with fluorescent dyes, and the detection is effected by measurement of the change in fluorescence after the probe has bound to the sequence tag.

In a preferred embodiment, the probe belongs to the class of the dual-labeled probes. According to the invention these are oligonucleotides which are coupled both with a reporter fluorophor and also with a quencher. The nucleotides at the 5′ end of the probe are complementary to those at the 3′ end, so that a characteristic secondary structure can form. An example of such a characteristic secondary structure is a so-called hairpin structure, such as is for example to be found in molecular beacons. In the hairpin structure state, the probe emits very little or no fluorescence owing to the small distance between fluorophor and quencher therein. Through the attachment of the probe to the sequence tag, the distance between fluorophor and quencher is increased, as a result of which an increase in the fluorescence emission can be observed.

A further example of a dual-labeled probe according to the invention comprises a hydrolysis probe, for example a TagMan® probe, in which the probe is degraded at the 5′ end during the synthesis of the reverse strand and fluorophor and quencher are thus brought to a greater distance from one another.

Further embodiments of dual-labeled probes are familiar to those skilled in the art.

In a further preferred embodiment, the detection of the sequence tag by a probe is effected by means of melting curve analysis. In a melting curve analysis, the double-stranded nucleic acid is melted by slowly and continuously raising the temperature. At a melting temperature specific for the hybrid of probe and sequence tag, the double strand denatures to two single-stranded molecules. Since single-stranded nucleic acids have different absorption properties at 260 nm from double-stranded ones, a melting curve analysis can be performed at 260 nm without dye labels on the probe or substances intercalating into double-stranded nucleic acids being necessary for the detection.

In an especially preferred embodiment, the melting curve analysis takes place by means of dyes specific for double-stranded DNA, for example by means of intercalating fluorescent dyes. At the melting temperature, the double strand which is formed from the probe and the sequence tag denatures to two single-stranded molecules. In the process, the fluorescent dye is released, and a decrease in fluorescence is recorded. Examples of fluorescent dyes intercalating into double-stranded DNA are SYBR® Green and EvaGreen®. Many further double stranded DNA-specific fluorescent dyes which are suitable for the melting curve analysis according to the invention are known to those skilled in the art.

In a further especially preferred embodiment, the melting curve analysis is effected by means of a labeled probe. Examples of such labeling are fluorescent dyes or quantum dots. Those skilled in the art are familiar with other possibilities for labeling the probe.

In a preferred embodiment, more than one sequence tag is detected in a multiplexing approach.

In a preferred embodiment, the sequence of the sequence tag differs in its nucleotide sequence to the extent that in the multiplexing approach several different probes can bind to different sequence tags and thus several different sequence tags can be detected simultaneously in one reaction vessel.

In an especially preferred embodiment, each sequence tag differs in its nucleotide sequence and/or in its melting temperature with its own complementary probe, so that in the multiplexing approach several different sequence tags can be detected simultaneously in step d) on the basis of different melting temperatures.

Here the number of target nucleic acids analyzable in parallel in a multiplexing approach follows from the number of the different melting temperatures which can be distinguished from one another by the appropriate analytical instrument, combined with the number of the different fluorescent dyes which can be distinguished from one another at different wavelengths by the particular analytical instrument. If two sequence tags have the same melting temperature, then these can nonetheless be specifically detected together and distinguished from one another in a multiplexing approach, if their own respective probes have different fluorescent dyes which emit the fluorescence at different wavelengths, so that these can be detected in different fluorescence channels. Conversely, probes which hybridize with different sequence tags can have the same fluorescent labeling if the melting temperatures of these probes with their own sequence tags differ. These are then detected in the same fluorescence channel, but can nonetheless be distinguished from one another through their different melting temperatures.

In principle, all instruments which are capable of detecting changes in the relevant wavelength region are suitable for the detection of the sequence tag by means of a suitable probe.

In the case of a melting curve analysis without the addition of dyes, instruments which are capable of measuring absorption differences in a wavelength region of about 260 nm are suitable for this. These instruments for example include UV spectrophotometers. Further suitable instruments are familiar to those skilled in the art.

If the detection of the sequence tag is effected by means of a fluorescent dye, then all instruments which are able to measure the fluorescence of the relevant wavelength are suitable for the detection. These include for example real-time PCR cyclers or spectrophotometers. Further suitable technical instruments are well known to those skilled in the art.

### FIGURES

FIG. 1 shows the development of fluorescence of the isothermal amplification as a function of time. The number of cycles is given on the x-axis, and the logarithm of the fluorescence on the y-axis. The dashed line shows the boundary of the background fluorescence.

FIG. 2 shows on the left-hand side the melting curve analysis after the isothermal amplification in the presence of PCR amplification products both from C. glutamicum and also E. coli. The temperature in ° C. is shown on the x-axis, and the relative fluorescence on the y-axis. In addition, on the right-hand side the relative fluorescence when either only PCR amplification products from C. glutamicum or E. coli were used in the isothermal amplification is also shown as a control.

### EXAMPLES

The following examples serve for illustration of the invention, without this being restricted to the practical examples.

Primer sequences:
eCG-FWD:
5′-GCTCCAGCCACCCAAAAC
eCG-REV:
5′-GGCTTCATCGACAGTCTGACGACCGACTCAACCACTAATGCGTCGTC
eCG-PRO:
5′-6FAM-GGCTTCATCGACAGTCTGAC-BHQ1
eCG-ctrl:
5′-GTCAGACTGTCGATGAAGCC
EOF:
5′-ATGCTACCCCTGAAAAACTC
eEC-REV:
5′-TTTACTTCTTTGCGTTATGTCTCTGACTCGCTTGAACTGATTTCCTC
eEC-PRO:
5′-6FAM-TTTACTTCTTTGCGTTAT-BHQ1

### Example 1

### Detection of a Part Region of the Nucleotide Sequence of the Polyketide Synthase of Corynebacterium glutamicum

By means of the primer pair eCG-FWD and eCG-REV (see above for sequences), 2 pmol of each, a part region of the nucleotide sequence of the polyketide synthase was amplified from 10 to 40 ng of genomic DNA from Corynebacterium glutamicum in a PCR. The reaction mixture has a volume of 20 μl, and the HotStar-Taq DNA polymerase (QIAGEN) was used for the amplification. The reaction conditions were as follows:

1) 15 min 95° C.

2) 35 cycles:
15 secs 55° C.
40 secs 72° C.
15 secs 94° C.

3) 2 mins 72° C.

4) 5 mins 98° C.

After the PCR amplification, 5 μl of the amplification product of the target nucleic acid were inserted into the reaction for the isothermal amplification of the sequence tag (overall volume 10 μl). In addition, desoxyribonucleotides (5 mM of each), 3 U of N.BstNBI (NEB), 0.2 U of Vent exo⁻ (NEB) and 2 pmol of the oligonucleotide eCG-PRO were further added to this reaction. The reaction mixture was heated to 56° C. and the temperature maintained constant at this temperature for the whole reaction time (45 mins). Every 30 seconds, the fluorescence was measured.

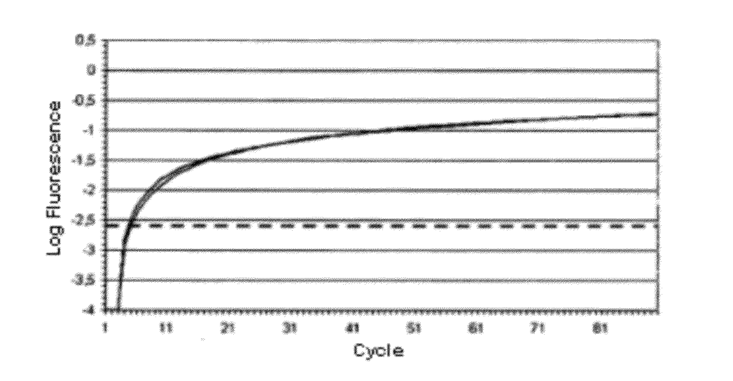

FIG. 1 shows the rise in fluorescence in real time. Thus it can be seen from this figure that very quickly, after 90 seconds, the fluorescent signal lies above the fluorescence noise and the sequence tag is thus already detectable after 90 seconds of the isothermal amplification. The fluorescence curve reaches a plateau after about 40 minutes, so that after this time no further sequence tags can any longer be additionally detected. The detection of a target nucleic acid is thus possible rapidly and efficiently with the method according to the invention. This detection takes place very rapidly, and signals clearly lying above the background can be detected within three minutes.

### Example 2

### Simultaneous Detection of a Part Region of the Nucleotide Sequence of the Polyketide Synthase of Corynebacterium glutamicum and a Part Region of the Nucleotide Sequence of Intimin from Escherichia coli

The amplification of the nucleotide sequence of the polyketide synthase was performed as described in example 1, and the amplification of the nucleotide sequence of intimin was performed in a separate PCR. The reaction parameters were the same as described in example 1, except that in the case of the amplification of the nucleotide sequence of intimin 10 to 40 ng of genomic DNA from Escherichia coli were used and the primers EOF and eEC-REV (see above for sequences) were used for the amplification.

After the PCR amplification, 2.5 μl each of the amplification products in one reaction vessel were inserted into the reaction for the isothermal amplification of the two sequence tags for 45 min. The reaction parameters were the same as stated in example 1, except that a further 2 pmol of the oligonucleotide eEC-PRO (see above for sequence) were inserted into the reaction.

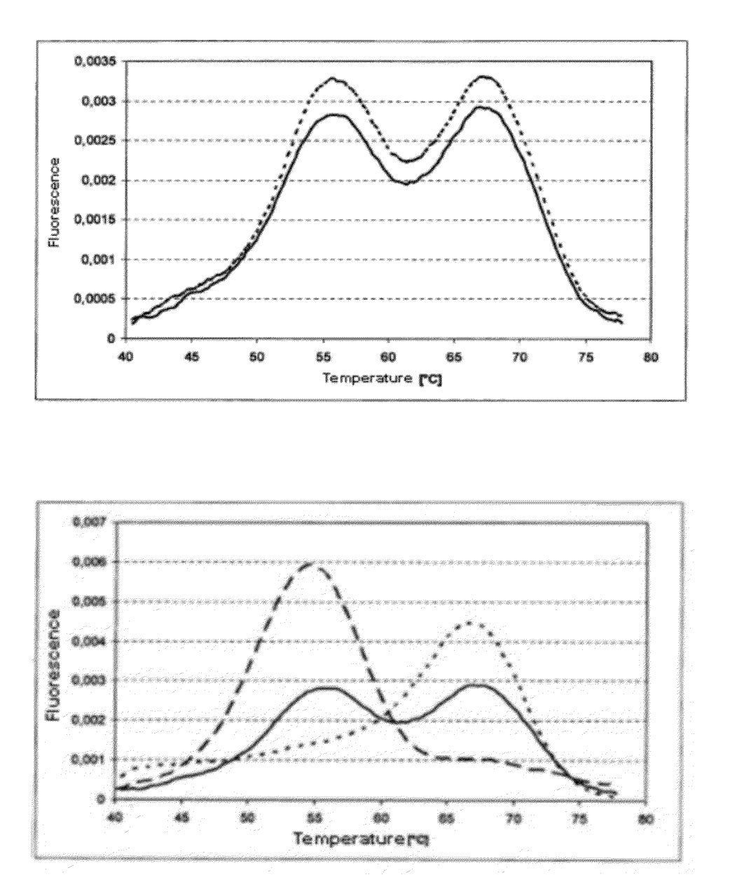

The melting curve was conducted following the isothermal amplification in the Real-Time Cycler (MJ Research Opticon, Bio-Rad). FIG. 2 shows the melting curve of the sequence tags with the probes respectively targeted on them. Since the melting temperature of the two sequence tags with the probes respectively targeted on them differs, the two sequence tags can be detected simultaneously by a melting curve analysis. The melting curve in FIG. 2 (left) clearly shows two peaks, one at about 56° C., the second at about 68° C., which demonstrates the presence of both sequence tags in the isothermal amplification reaction. For the control, FIG. 2 (right), in addition the melting curves after the isothermal amplification were investigated when only the amplification product of the PCR of Corynebacterium glutamicum or only the amplification product of the PCR of Escherichia coli respectively was inserted into the isothermal amplification.

Thus it could be shown that the method according to the invention is also outstandingly suitable for multiplex detections.

1. A method for detecting target nucleic acids, comprising the following process steps:
a) primer-mediated amplification of at least one target nucleic acid, wherein at least one of the primers used for the amplification is a sequence tag primer which has a non-hybridizing part at its 5′ end, wherein this non-hybridizing part has a first sequence which during the amplification creates a cleavage site for a nicking endonuclease on a newly synthesized strand complementary to it, and furthermore 5′ from this first sequence has a second sequence which during the amplification creates a sequence tag on a newly synthesized strand complementary to it;
b) contacting the at least partly double-stranded amplification product from step a) with nucleotides, a nicking endonuclease and a polymerase, wherein the polymerase has a strand displacement activity and no 5′→3′ exonuclease activity;
c) isothermal amplification of the sequence tag created in step a) by single or multiple repetition of a cycle having the following steps
i) insertion of a nick at the cleavage site inserted in step a) by means of the endonuclease from step b), and
ii) filling of the nick beginning at the free 3′ end created in step i) with complementary nucleotides by means of the polymerase from step b) with simultaneous displacement of the sequence tag from the double strand; and
d) specific detection of the sequence tag amplified in step c).

2. The method as claimed in claim 1, wherein in step a) two or more target nucleic acids are amplified.

3. The method as claimed in claim 2, wherein for each of the target nucleic acids in step a) at least one specific sequence tag primer is used in each case.

4. The method as claimed in claim 1, wherein the amplification of the target nucleic acid from step a) is effected by means of PCR.

5. The method as claimed in claim 1, wherein the amplification of the target nucleic acid from step a) is effected by means of isothermal amplification.

6. The method as claimed in claim 1, wherein the nicking endonuclease from step b) is one or more selected from the group consisting of Nt.BstNBI, Nt.BspQI, Nb.BbvCI, Nb.Bsml, Nb.BsrDI, Nb.Btsl, Nt.Alwl, Nt.BbvCI, Nt.CviPII, Nt.BsmAI, Nb.Bpu10I and Nt.Bpu10I.

7. The method as claimed in claim 1, wherein the polymerase from step b) is one or more selected from the group consisting of Vent exo⁻, Deep Vent exo⁻, Bst exo⁻, the Klenow fragment of DNA polymerase I, Phi29 DNA polymerase and 9° Nm DNA polymerase.

8. The method as claimed in claim 1, wherein the detection from step d) is effected by means of a probe targeted on the sequence tag.

9. The method as claimed in claim 8, wherein the probe is fluorescence-labeled.

10. The method as claimed in claim 8, wherein the detection is effected by means of melting curve analysis.

11. The method as claimed in claim 9, wherein the fluorescence-labeled probe is a dual-labeled probe.

12. The method as claimed in claim 1, wherein the target nucleic acid is genomic DNA, plasmid DNA, viral DNA, mitochondrial DNA or plastid DNA or a fragment of one or more thereof.

13. The method as claimed in claim 1, wherein the polymerase is a DNA polymerase and the nucleotides are desoxyribonucleotides.

14. The method as claimed in claim 1, wherein the amplification from step a) is effected by means of nested primers and the sequence tag primer is a primer of the inner primer pair.
